(19) United States
(12) Patent Application Publication
(10) Pub. No.: US 2025/0208116 A1
(43) Pub. Date: Jun. 26, 2025

(54) A COMPOSITION FOR COATING A CUVETTE AND A METHOD OF MAKING A COMPOSITION FOR COATING A CUVETTE

(71) Applicant: Entia Limited, London (GB)

(72) Inventors: Laura Barrera Moreno, London (GB); Christopher Barrows, London (GB); Thuy-Vi Truong, London (GB)

(21) Appl. No.: 18/847,902

(22) PCT Filed: Mar. 17, 2023
(86) PCT No.: PCT/GB2023/050644

§ 371 Date: Sep. 17, 2024

(30) Foreign Application Priority Data
Mar. 18, 2022 (GB) 2203783.2

(51) Int. Cl. G01N 33/52; G01N 21/03; G01N 33/50

(52) U.S. Cl. CPC G01N 33/52; G01N 21/03; G01N 33/5002

(57) ABSTRACT

The present invention relates to a composition and a method of making a composition. In particular, the present invention relates to a composition for coating a cuvette and a method of making a composition for coating a cuvette.

### FIELD OF THE INVENTION

The present invention relates to a composition and a method of making a composition. In particular, the present invention relates to a composition for coating a cuvette and a method of making a composition for coating a cuvette.

### BACKGROUND OF THE INVENTION

Analytical devices that determine an optical property of a sample contained within a cuvette are known. In such devices, the cuvette is at least partially filled with a sample for analysis and then placed into the device. Optical (or other) radiation is passed through the sample to allow properties of the sample, such as the presence of particular components, to be measured.

A common sample examined in analytical devices is a biological sample such as a blood sample. The analysis of blood is used in a wide range of medical monitoring methods, such as in monitoring the progress of systemic anti-cancer therapy. For example, an optical analysis device can analyse a blood sample to determine the presence, absence and/or amount of white blood cells, such as granulocytes and agranulocytes, which the presence, absence and/or amount of is often used in monitoring the progress of systemic anti-cancer therapy. Analysis of a blood sample typically requires a sample to be centrifuged first. During centrifugation, the blood is separated into layers. The layers being: (a) an erythrocyte layer which contains red blood cells; (b) a buffy coat layer which contains white blood cells (such as granulocytes and agranulocytes); (c) a platelet (thrombocyte) layer; and, (d) a plasma layer. The blood is then analysed with optical (or other radiation) to obtain an image of the layers. A typical source of optical radiation is LED light. The pixel count of each layer from the image can then be calculated to determine the cell count of each layer.

A cuvette is a disposable part of an analytical device, and commonly a new cuvette is required for each measurement. It is important that a user can load a sample into the cuvette quickly and easily. It is also important that the cuvette can be manufactured at a low cost, can be easily loaded with a sample and secured on the holder of the optical analysis device, and, can be readily removed from the holder once analysis is complete. Examples of known types of cuvettes can be seen in, for example, GB2555403A, EP2096444A1, EP1055112A1, WO2007/008137, U.S. Pat. No. 6,365,104B1 and WO2018078324A1 (the disclosures of which are each incorporated herein by reference).

The cuvette in which the sample is contained can be coated with a composition. The purpose of the composition is three-fold. The composition (a) aids the collection of the sample by the cuvette; (b) ensures that once the sample is collected the sample remains processable; and, (c) aids the production of a clear image of the sample. Traditionally, the composition in which the cuvette is coated comprises potassium oxalate, acridine orange, sodium heparin and ethylenediaminetetraacetic acid dipotassium salt dehydrate. Examples of known compositions used in cuvettes can be seen in, for example, U.S. Pat. No. 6,365,104B1 (the disclosure of which is incorporated herein by reference).

There is a need for improved compositions that can be used with cuvettes for sample analysis. In particular, there is a need for improved compositions that provide a clear image of a biological sample in a cuvette. In particular, there is a need for improved compositions that provide a clear image of the buffy coat layer of a blood sample in a cuvette.

### SUMMARY OF THE INVENTION

The present invention relates to a composition and a method of making a composition. In particular, the present invention relates to a composition for coating a cuvette and a method of making a composition for coating a cuvette.

Representative features of the present invention are set out in the following clauses, which stand alone or may be combined, in any combination, with one or more features disclosed in the text and/or figures of the specification.

The present invention is as set out in the following clauses:

1. A composition for analysing biological samples comprising:
a collection solution; and
an imaging solution;
   wherein the collection solution comprises:
an anticoagulant agent;
an agglutination agent; and,
a wetting agent.

2. The composition of clause 1, wherein the collection solution comprises:
an anticoagulant agent at from 5 to 25 mg/ml;
an agglutination agent at from 5 to 25 mg/mL; and a wetting agent at from 0.05 to 0.25 mg/mL.

3. The composition of clause 1 or clause 2, wherein the collection solution and/or the imaging solution further comprise(s) water (optionally, wherein the water is distilled water), or, alcohol solvents.

4. The composition of any one of clauses 1 to 3, wherein the collection solution comprises a first anticoagulant agent and a second anticoagulant agent, wherein the first anticoagulant agent and the second anticoagulant agent are different.

5. The composition of any one of clauses 1 to 4, wherein the anticoagulant agent(s) is/are one or more of ethylenediaminetetraacetic acid dipotassium salt dehydrate, potassium oxalate, ammonium-potassium oxalate, heparin, citrate, hirudin or any combination thereof.

6. The composition of any one of clauses 1 to 5, wherein the agglutination agent is one or more of polyvinylpyrrolidone, proteolytic enzymes such as bromelain, pepsin and/or trypsin, polyethylene glycol or any combination thereof.

7. The composition of any one of clauses 2 to 6, wherein the wetting agent is one or more of a well-known surfactant (that is ionic, non-ionic and/or cationic), pluronic P-123, silwet L600, fatty alcohol ethoxylates, alkyl phenol ethoxylates, fatty acid alkoxylates or any combination thereof.

8. The composition of any one of clauses 1 to 7, wherein the collection solution comprises:
(a) an anticoagulant agent at from 5 to 25 mg/mL, or, from 7.5 to 20 mg/mL, or, from 10 to 15 mg/mL, or, 13 mg/LI; and/or,
(b) an agglutination agent at from 5 to 25 mg/mL, or, from 7.5 to 20 mg/mL, or, from 9 to 11 mg/mL, or, 10 mg/mL; and/or,
(c) a wetting agent at from 0.050 to 0.250 mg/mL, or, from 0.075 to 0.120 mg/mL, or, from 0.900 to 0.125 mg/mL, or, 0.1 mg/mL.

9. The composition of any one of clauses 1 to 8, wherein the collection solution comprises a first anticoagulant agent at from 2 to 10 mg/mL, or, from 4 to 8 mg/mL, or, from 5 to 7 mg/mL, or, at 6 mg/mL, and, a second anticoagulant agent at from 3 to 11 mg/mL, or, from 5 to 9 mg/mL, or, from 6 to 8 mg/mL, or, 7 mg/ml; wherein the first anticoagulant agent and the second anticoagulant agent are different.

10. The composition of any one of clauses 1 to 9, wherein the collection solution comprises, or consists of, ethylenediaminetetraacetic acid dipotassium salt dehydrate, potassium oxalate, polyvinylpyrrolidone, pluronic P-123 and water; optionally wherein the water is distilled water.

11. The composition of any one of clauses 1 to 10, wherein the collection solution comprises:
(a) ethylenediaminetetraacetic acid dipotassium salt dehydrate agent at from 3 to 11 mg/mL, or, from 5 to 9 mg/mL, or, from 6 to 8 mg/mL, or, 7 mg/ml;
(b) potassium oxalate at from 2 to 10 mg/mL, or, from 4 to 8 mg/mL, or, from 5 to 7 mg/mL, or, 6 mg/mL;
(c) polyvinylpyrrolidone at from 5 to 25 mg/mL, or, from 7.5 to 20 mg/mL, or, from 9 to 11 mg/mL, or, 10 mg/mL; and,
(d) pluronic P-123 at from 0.050 to 0.250 mg/mL, or, from 0.075 to 0.120 mg/mL, or, from 0.900 to 0.125 mg/mL, or, 0.1 mg/mL;
   the balance being water; optionally, wherein the water is distilled water.

12. The composition of any one of clauses 1 to 11, wherein the collection solution comprises ethylenediaminetetraacetic acid dipotassium salt dehydrate agent at 7 mg/mL; potassium oxalate at 6 mg/mL; polyvinylpyrrolidone at 10 mg/mL; and, pluronic P-123 at 0.1 mg/ml; the balance being water; optionally, wherein the water is distilled water.

13. The composition of any one of clauses 1 to 12, wherein the imaging solution comprises:
an imaging agent.

14. The composition of clause 13, wherein the imaging solution further comprises water (optionally, wherein the water is distilled water), or, alcohol solvents.

15. The composition of clause 13 or clause 14, wherein the imaging agent is one or more of acridine orange, quaternary cationic metachromatic dyes such as Greifswalder's blue, blue borrel, rhodanile blue, toluylene blue, night blue, Hofmann's violet, basic orange 21, permanent dyes such as cell permanent cyanine dyes, SYTO dyes, oxayne dyes, phenanthridines (intercalating) dyes, indoles dyes, imidazole dyes or any combination thereof.

16. The composition of any of clauses 13 to 15, wherein the imaging agent has a concentration at from 0.01 to 0.030 mg/mL, or from 0.015 to 0.025 mg/mL, or from 0.019 to 0.021 mg/mL, or, 0.020 mg/mL.

17. The composition of any one of clauses 1 to 16, wherein the imaging solution comprises, or consists of, acridine orange and water; optionally, wherein the water is distilled water.

18. The composition of any of clauses 1 to 17, wherein the imaging solution comprises acridine orange at from 0.01 to 0.030 mg/mL, or, from 0.015 to 0.025 mg/mL, or, from 0.019 to 0.021 mg/mL, or, 0.020 mg/mL; the balance being water; optionally, wherein the water is distilled water.

19. The composition of any one of clauses 1 to 18, wherein the imaging solution comprises acridine orange at 0.020 mg/mL; the balance being water; optionally, wherein the water is distilled water.

20. The composition of any one of clauses 1 to 19, wherein the imaging solution has a pH of from 3 to 6, or, from 3.5 to 4.5, or, 4.

21. The composition of any one of clauses 1 to 20, wherein the composition is a two-part composition, the collection solution and the imaging solution being separate prior to combining the collection solution and the imaging solution when analysing a biological sample.

22. The composition of any one of clauses 1 to 21, wherein the collection solution and imaging solution are combined to form one solution.

23. A method of making the composition of any one of clauses 1 to 22, the method comprising the following steps:
(a) providing a collection solution; and
(b) providing an imaging solution;
   wherein the collection solution comprises:
an anticoagulant agent;
an agglutination agent; and,
a wetting agent.

24. The method of making the composition of clause 23, wherein step (a) further includes the following steps:
(c) making a stock solution for the anticoagulant agent, the agglutination agent and the wetting agent; and
(d) combining the stock solutions.

25. The method of making the composition of clause 23 or clause 24, wherein step (b) further includes the following steps:
(e) providing an imaging agent;
(f) making a stock solution for the imaging agent; and
(g) diluting the stock solution of the imaging agent

26. The method of making the composition of clauses 23 to 25, wherein the collection solution and imaging solution are as defined in clauses 1 to 22.

27. The composition of any one of clauses 1 to 22, for use on a glass capillary tube or a cuvette; optionally, a disposable cuvette.

28. The composition of clause 27, wherein the cuvette comprises a collection chamber and an analysis chamber, and, wherein the collection chamber is coated (fully or partially) in the collection solution and the analysis chamber is coated (fully or partially) in the imaging solution.

29. The composition of any one of clauses 1 to 22, for use in the analysis of biological samples, optionally, a blood biological sample.

### DETAILED DESCRIPTION

Embodiments of the invention are described below with reference to the accompanying drawings. The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

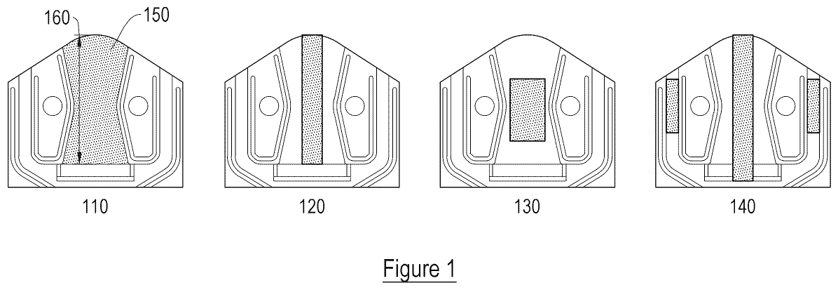

FIG. 1 depicts acceptable and unacceptable coatings of the collection solution on a collection chamber of half of a cuvette.

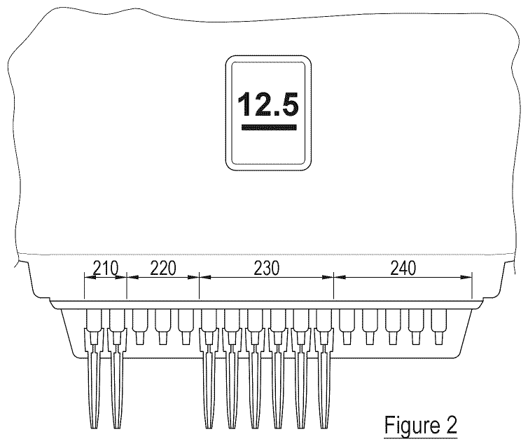

FIG. 2 depicts the GRIPTIP spacing used in a multichannel pipette.

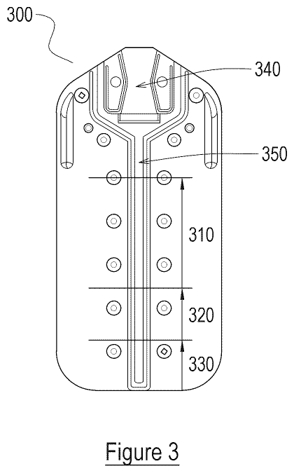

FIG. 3 shows the favoured placing of the imaging solution on an analysis chamber of half of a cuvette.

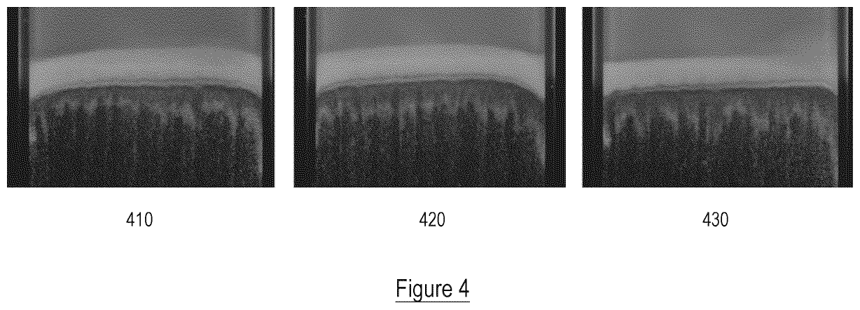

FIG. 4 shows images obtained when analysing the effect of the concentration of the agglutination agent on the quality of the image obtained of a blood sample.

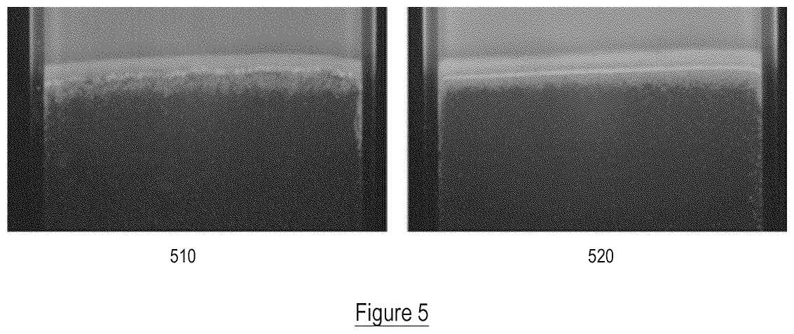

FIG. 5 shows images obtained of a blood sample inside a cuvette coated in the composition of the present invention, and a blood sample inside a cuvette coated with a different composition (not according to the present invention).

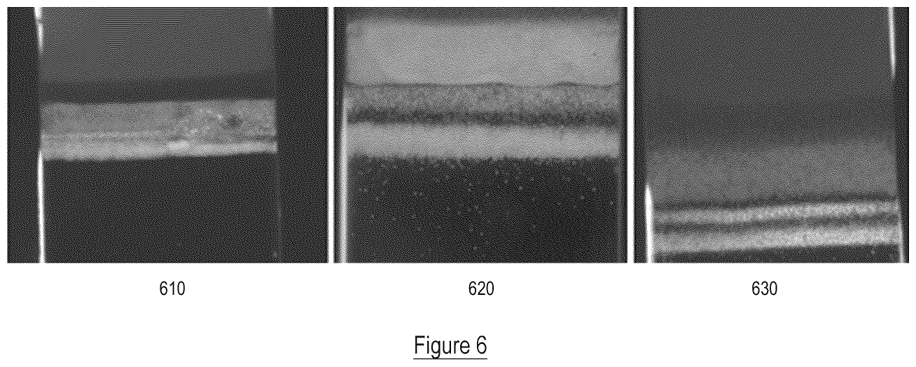

FIG. 6 shows images obtained when the entire length of a middle wall of an analysis chamber of half of a cuvette was coated in the imaging solution.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Some of the terms used to describe the present invention are set out below:

“Acridine orange” refers to an imaging dye. Acridine orange causes cells to fluoresce upon exposure to optical radiation. Typically, acridine orange is excited at a wavelength of from 200 to 460 nm. The mechanism by which acridine orange can differentiate cells is called metachromasia. With metachromasia acridine orange binds to different components in a sample upon excitation, creating peaks at different wavelengths in a spectrum. For example, when acridine orange binds to DNA, the cells will fluoresce green at a maximum emission of 530 nm upon excitation with blue light. When acridine orange binds to RNA, the resultant combination condenses (i.e., transitions to a solid state) and then precipitates. The resultant components will phosphoresce or luminesce red at a maximum emission of 640 nm upon excitation with blue light. The concentration of acridine orange also has an impact on the colour emission of the sample because concentration will affect the form of genetic material available. It is well-known that acridine orange is less sensitive in the RNA selection than DNA. For example, a high acridine orange concentration leads to denaturation of DNA whilst also colouring the DNA red. This is because a high concentration of acridine orange causes the DNA to become single stranded. Therefore, a high acridine concentration will cause a high amount of red emission. To the contrary, a low acridine orange concentration causes incomplete RNA denaturation and results in a portion of RNA staining green. Therefore, a low acridine orange concentration will cause a high amount of green emission. Furthermore, the colouring arising from using acridine orange binding to cells depends on the structure of the cell and can aid the differentiation of different cells. For example, granulocyte cells stain an orange colour in the granules and a red colour in the nuclei causing granulocyte cells to emit significantly in the red spectrum, whereas agranulocyte cells stain a yellow-green colour in their nuclei and a red colour in their cytoplasm, causing the agranulocyte cells to emit in the green spectrum.

“Agglutination agent” refers to a material that aids the clumping together of particles. For example, an agglutination agent can aid the clumping of cells such as bacteria or red blood cells. In particular, an agglutination agent can clump together red blood cells by acting directly on the red blood cells, for example but not limited to, by reducing the repulsive force between red blood cells. Examples of agglutination agents include, but are not limited to lectin proteins, or, hemagglutinins proteins, or, polyvinylpyrrolidone, a solution containing polyvinylpyrrolidone K12, proteolytic enzymes such as bromelain, pepsin and/or trypsin, or polyethylene glycol.

“Agranulocytes” refers to a type of white blood cell that has no distinct granules. Examples of agranulocytes include, but are not limited to, lymphocytes and monocytes.

“Alcohol solvents” refers to a solvent which is alcohol based. Alcohol solvents includes, but is not limited to, benzyl alcohol, 1,4-butanediol, 1,2,4-butanetriol, butanol, 1-butanol, 2-butanol, tert-butyl alcohol, and ethanol.

“Anticoagulant agent” refers to a material that prevents the clotting of blood. The anticoagulant ensures that a blood sample is not significantly changed prior to the analysing the sample. Examples of anticoagulant agents include, but are not limited to, ethylenediaminetetraacetic acid dipotassium salt dehydrate, potassium oxalate, ammonium-potassium oxalate, heparin, citrate and hirudin.

“Buffy coat” refers to a fraction of an anticoagulated blood sample. The buffy coat contains most of the white blood cells and platelets in a centrifuged blood sample.

“Collection solution” refers to a solution comprising an anticoagulant, an agglutination agent, and a wetting agent. The collection solution can additionally comprise water. The purpose of the collection solution is typically to improve the ease and efficiency in which a sample can be taken up by an analytical device, such as a cuvette.

“Cuvette” refers to a container used in analytical devices and in particular a container for analysis of a sample under centrifugation. An example of a cuvette is described in GB2555403A and UK application GB2108004.9 (the disclosure of which are incorporated herein by reference).

“Entia Liberty™ device” refers to a device manufactured by Entia Ltd. which uses optical radiation to measure the concentration of components in a sample. In particular, the device is used to measure the presence (by measuring the amount) of white blood cells in a blood sample taken from a patient undergoing monitoring for the progress of systemic anti-cancer therapy.

“Fluid” refers to any material that has no fixed shape and yields easily to external pressure. An example of a fluid includes, but is not limited to, a blood sample.

“Granulocytes” refers to a type of white blood cell that has small granules. These granules contain proteins. Examples of granulocytes are neutrophils, eosinophils, and basophils.

“Homogeneous” refers to a substance that has uniform composition and properties throughout.

“Imaging solution” refers to a solution comprising an imaging agent. Examples of imaging agents include, but are not limited to, acridine orange, quaternary cationic metachromatic dyes such as Greifswalder's blue, blue borrel, rhodanile blue, toluylene blue, night blue, Hofmann's violet, basic orange 21, permanent dyes such as cell permanent cyanine dyes, SYTO dyes, oxayne dyes, phenanthridines (intercalating) dyes, indoles dyes or imidazole dyes. The imaging solution can additionally comprise water. The purpose of the imaging solution is to ensure that an image of a sample to be analysed can be obtained by optical (or another source) radiation.

“Multichannel pipette” refers to an electronic device used in laboratories to accurately measure and fill numerous vials with liquid at once. Examples of multichannel pipettes include, but are not limited to, Eppendorf Research plus Multichannel pipette, the CappAero Multichannel pipette and the VIAFLO multichannel pipette. Preferably, a multichannel pipette with 4 mm or less distance between tips and that can dispense at least 0.5 μL of liquid can be used.

“Mixture” refers to a material made by mixing two or more different components together. The mixture may be uniform or may not be uniform throughout.

“Room temperature” refers to a temperature of from 18 to 25° C., preferably from 20 to 22° C., preferably 20° C.

“Sample” refers to a substance for analysis. The sample is analysed in a fluid phase.

“Wetting agent” refers to refers to a material that increases the spreading and penetrating properties of a liquid by lowering the surface tension of the liquid. An example of a wetting agent includes, but is not limited to, well known surfactants (that are ionic, non-ionic and/or cationic), pluronic P-123 (otherwise known as poly(ethylene glycol)-block-poly(propylene glycol)-block-poly(ethylene glycol)), silwet L600, fatty alcohol ethoxylates, alkyl phenol ethoxylates or fatty acid alkoxylates.

### Composition

The present invention provides a composition. In particular, the composition is for coating the whole or a part of a cuvette. The composition is improved over existing compositions for coating a cuvette. The composition of the present invention has at least the following benefits: improved ease and efficiency for uptake of a sample by a cuvette; improved imaging of the sample held within the cuvette; improved imaging of the sample held within the cuvette by allowing visualisation of the different layers of the buffy coat of a blood sample.

The composition comprises a collection solution and an imaging solution.

The collection solution comprises an anticoagulant, an agglutination agent and a wetting agent. The collection solution can also comprise water or any suitable organic or inorganic solvent such as alcohol solvents.

The anticoagulant agent includes one or more of ethylenediaminetetraacetic acid dipotassium salt dehydrate, potassium oxalate, ammonium-potassium oxalate, heparin, citrate, hirudin or any combination thereof.

The agglutination agent includes one or more of polyvinylpyrrolidone, a solution containing polyvinylpyrrolidone K12, proteolytic enzymes such as bromelain, pepsin and/or trypsin, polyethylene glycol or any combination thereof. Advantageously, the inclusion of an agglutination agent in the collection solution reduces blood relaxation after a blood sample is centrifuged in a cuvette that comprises the collection solution. This advantageously lengthens the time period available for imaging the sample. Further advantageously, the agglutination agent can act as a plasma expander to a blood sample, causing the plasma volume and sedimentation rate to increase whilst decreasing haematocrit.

The wetting agent includes one or more of pluronic P-123, silwet L600, fatty alcohol ethoxylates, alkyl phenol ethoxylate, fatty acid alkylates or any combination thereof.

Preferably, the pH of the collection solution is acidic. Preferably, the pH of the collection solution is from 3 to 6, or, from 3.5 to 4.5, or, 4 (plus or minus 0.2).

The imaging solution comprises an imaging agent. The imaging solution can also comprise water or any suitable organic or inorganic solvent such as alcohol solvents.

The imaging agent includes one or more of acridine orange, quaternary cationic metachromatic dyes such as Greifswalder's blue, blue borrel, rhodanile blue, toluylene blue, night blue, Hofmann's violet, basic orange 21, permanent dyes such as cell permanent cyanine dyes, SYTO dyes, oxayne dyes, phenanthridines (intercalating) dyes, indole dyes, imidazole dyes or any combination thereof. Advantageously, the dyes are positively charged and are therefore capable of crossing the cell membrane of a blood cell. The dyes can therefore chemically stain the blood cells. Visually staining the blood cells enables visualisation of the blood cells. Further advantageously, the dyes can be used over a large pH range, temperature range and the staining properties of the dyes are not affected by a concentration variation. The dyes are fixed to a position (because the dyes are dried on the surface of the cuvette) and do not require the cells to be fixed in a position. Instead, the cells can be free flowing. Free flowing cells are desirable for imaging, because when cells are fixed the cells tend to lose important information, such as biochemical information.

Preferably, the pH of the imaging solution is acidic. Preferably the pH of the imaging solution is from 3 to 6, or, from 3.5 to 4.5, or, 4 (plus or minus 0.2).

### Cuvette

In some examples, the present invention also relates to a cuvette fully or partially coated in the composition.

In some examples, the cuvette is the cuvette described in the UK patent application GB2108004.9 filed on 4 Jun. 2021 (the disclosure of which is incorporated herein by reference).

In some examples, the cuvette is formed from two halves (called a cuvette half hereon in), which are manufactured separately and attached to one another. One half may include an upper surface of a cuvette, with the other half including the lower surface thereof. One or both halves may be formed with indentations on their inner surfaces so that, when the two halves are fixed together one or more chambers exist within the main body of the cuvette. Preferably, the main body of the cuvette has a sample chamber formed therein, the sample chamber communicating with the exterior of the cuvette by an opening formed in the exterior of the main body, wherein the sample chamber comprises a collection chamber and an analysis chamber. FIG. 3 shows a cuvette half (300) having a collection chamber (340) and analysis chamber (350). The collection chamber has a first end in fluid communication with the opening and a second end in fluid communication with the analysis chamber. The analysis chamber has a first end which is in fluid communication with the collection chamber, and a second, closed, end. The analysis chamber is in fluid communication with the exterior of the cuvette only through the collection chamber. However, it should be understood that the invention is not limited to this method of manufacture of the cuvette, and the cuvette may be formed in any other suitable or convenient manner.

In some examples, indentations are formed on the inner surface of one half of the cuvette, with the inner surface of the other half being substantially smooth and/or flat. In other examples, both halves may have indentations formed on their inner surfaces, and when the cuvette is assembled then at least some of the indentations align with each other to form the chambers. As discussed, the various chambers may be created by indentations formed on the inner sides of one or both of the halves of the cuvette. The skilled reader will appreciate that there are many other possibilities.

In some examples, the collection chamber and the analysis chamber have a depth which is the same or generally the same.

In some examples, the collection chamber has four opposing side walls which are parallel with each other. The collection chamber extends from a first end in fluid communication with the opening formed in the exterior of the main body and a second end in fluid communication with the analysis chamber. In some examples, the analysis chamber is preferably of consistent width along its length, having four opposing side walls which are parallel with each other. Preferably, when the cuvette is made from two cuvette halves wherein one cuvette half has indentations to form the chambers and the second cuvette half is substantially smooth or flat, the indentations on the one cuvette half form three of the four opposing side walls of the collection and analysis chambers and the substantially smooth or flat surface of the second cuvette half forms the fourth opposing wall. The analysis chamber is preferably of constant depth along its length. The analysis chamber extends towards the opposite end of the cuvette to the collection chamber and terminates at a closed end which does not allow any communication with the exterior of the cuvette. The closed end of the analysis chamber preferably has a squared-off shape, comprising an end surface which is at right-angles or substantially at right-angles to the length of the analysis chamber. The closed end can be called an endwall.

In some examples, the cuvette includes air vents which allow air to escape from the cuvette.

In some examples, the cuvette includes a transition chamber between the collection and analysis chambers. The function of the transition chamber is to prevent overfilling of the cuvette, whilst simultaneously avoiding cell damage and/or activation.

In some examples, the collection chamber is coated (fully or partially) with the collection solution of the present invention. In other examples, the analysis chamber is coated (fully or partially) with the imaging solution. In other examples, the collection chamber is coated (fully or partially) with the collection solution and the analysis chamber is coated (fully or partially) with the imaging solution.

### Method of Making the Composition

The present invention also provides a method for making a composition for coating a cuvette (fully or partially). The method includes the following steps:
(a) providing a collection solution; and
(b) providing an imaging solution.

The present invention also provides a method for making the collection solution. The method includes the following steps:
(a) providing an anticoagulant, an agglutination agent and a wetting agent;
(b) making a stock solution of the anticoagulant, the agglutination agent and the wetting agent; and
(c) combining the stock solutions.

During step (b), the stock solutions are made separately.

In some examples, the anticoagulant, the agglutination agent and the wetting agent are weighed separately into separate containers. An example of a preferred separate container is a disposable tray. Each container is cleaned prior to use with a cleaning agent, such as isopropyl alcohol.

In some examples, the anticoagulant, the agglutination agent and the wetting agent are separately transferred to separate vials and a solvent added to the vials. Preferably, the solvent used is water. Preferably, any suitable organic or inorganic solvent is used such as alcohol solvents. Preferably, the anticoagulant, the agglutination agent and the wetting agent are dissolved in the solvent by stirring the solution at from 700 rpm to 900 rpm, or, 800 rpm. Preferably, the anticoagulant, the agglutination agent and the wetting agent are dissolved in the solvent by stirring the solution for from 5 to 15 minutes, or, 10 minutes. Preferably, the anticoagulant, the agglutination agent and the wetting agent are dissolved in the solvent at a temperature of from 10 to 30° C., or, from 15 to 25° C.

In some examples, the collection solution comprises two anticoagulant agents: a first anticoagulant agent and a second anticoagulant agent. Preferably, the first anticoagulant agent and the second anticoagulant agent are different. Preferably, a stock solution comprising a first anticoagulant agent is made with the first anticoagulant agent at a concentration of from 25 to 75 mg/mL, or, from 35 to 65 mg/mL, or, from 45 to 55 mg/mL, or, 50 mg/mL. Preferably, a stock solution comprising a second anticoagulant agent is made with the second anticoagulant agent at a concentration of from 50 to 150 mg/mL, or, from 75 to 125 mg/mL, or, from 90 to 110 mg/mL, or, 100 mg/mL.

In some examples, a stock solution comprising the agglutination agent is made with the agglutination agent at a concentration of from 25 to 75 mg/mL, or, from 35 to 65 mg/mL, or, from 45 to 55 mg/mL, or, 50 mg/mL.

In some examples, a stock solution comprising the wetting agent is made with the wetting agent at a concentration of from 0.005 mg/ml to 0.015 mg/mL, or, from 0.0075 mg/ml to 0.0125 mg/mL, or, from 0.009 to 0.011 mg/mL, or, 0.01 mg/mL.

In some examples, a stock solution comprising a first anticoagulant agent is made with the first anticoagulant agent at a concentration of 50 mg/mL; a stock solution comprising a second anticoagulant agent is made with the second anticoagulant agent at a concentration of 100 mg/ml; a stock solution comprising a agglutination agent is made with the agglutination agent at a concentration of 50 mg/mL; and, a stock solution comprising the wetting agent is made with the wetting at a concentration of 0.01 mg/mL. Preferably, a stock solution comprising a potassium oxalate is made with potassium oxalate at a concentration of 50 mg/mL; a stock solution comprising ethylenediaminetetraacetic acid dipotassium salt dehydrate is made with ethylenediaminetetraacetic acid dipotassium salt dehydrate at a concentration of 100 mg/mL; a stock solution comprising polyvinylpyrrolidone is made with polyvinylpyrrolidone at a concentration of 50 mg/ml; and, a stock solution comprising pluronic P-123 is made with pluronic P-123 at a concentration of 0.01 mg/mL.

In some examples, the stock solutions can be stored for up to 4 weeks from the day in which the stock solution was made.

During step (c), the stock solutions are combined to form the collection solution.

In some examples, the collection solution comprises two anticoagulant agents: a first anticoagulant agent and a second anticoagulant agent. Preferably, the first anticoagulant agent and the second anticoagulant agent are different. Preferably, the collection solution comprises the first anticoagulant agent at from 2 to 10 mg/mL, or, from 4 to 8 mg/mL, or, from 5 to 7 mg/mL, or, 6 mg/mL. Preferably, the collection solution comprises the second anticoagulant agent at from 3 to 11 mg/mL, or, from 5 to 9 mg/mL, or, from 6 to 8 mg/mL, or, 7 mg/mL.

In some examples, the collection solution comprises the agglutination agent at from 5 to 25 mg/mL, or, from 7.5 to 20 mg/mL, or, from 9 to 11 mg/mL, or, 10 mg/mL.

In some examples, the collection solution comprises the wetting agent at from 0.050 to 0.250 mg/mL, or, from 0.075 to 0.120 mg/mL, or, from 0.900 to 0.125 mg/mL, or, 0.1 mg/mL.

In some examples, the collection solution comprises a first anticoagulant agent at 6 mg/ml; a second anticoagulant agent at 7 mg/mL; an agglutination agent at 10 mg/ml; and, a wetting agent at 0.1 mg/mL. Preferably, the collection solution comprises potassium oxalate at 6 mg/ml; ethylenediaminetetraacetic acid dipotassium salt dehydrate at 7 mg/ml; polyvinylpyrrolidone at 10 mg/ml; and, pluronic P-123 at 0.1 mg/mL

In some examples, the collection solution has a storage life of seven days. Once the seven days have passed, a fresh collection solution was prepared.

The present invention also provides a method for making the imaging solution. The method includes the following steps:
(a) providing an imaging agent; and
(b) making a stock solution for the imaging agent.

During step (b), the stock solution is made. In some examples, the imaging agent is a solid and is weighed into a container. An example of a preferred container is a disposable tray. The container is cleaned prior to use with a cleaning agent, such as isopropyl alcohol. In another example, the imaging agent is a liquid and the required amount of the imaging agent is measured into a measuring cylinder. The measuring cylinder is cleaned prior to use with a cleaning agent, such as isopropyl alcohol.

In some examples, the imaging agent is transferred to a vial and a solvent added to the vial. Preferably, the solvent used is water. Preferably, any suitable organic or inorganic solvent is used such as alcohol solvents. Preferably, the imaging agent is dissolved in the solvent by stirring the solution at from 700 rpm to 900 rpm, or, 800 rpm. Preferably, the imaging agent is dissolved in the solvent by stirring the solution at from 5 to 15 minutes, or, 10 minutes. Preferably, the imaging agent is dissolved in the solvent at a temperature of from 10 to 30° C., or, from 15 to 25° C.

In some examples, a stock solution comprising the imaging agent is made with the imaging agent at a concentration of from 0.01 to 0.030 mg/mL, or, from 0.015 to 0.025 mg/mL, or, from 0.019 to 0.021 mg/mL, or, 0.020 mg/mL.

In some examples, a stock solution comprising the imaging agent is made with the imaging agent at a concentration of 0.020 mg/mL. Preferably, a stock solution comprising acridine orange is made with acridine orange at a concentration of 0.020 mg/mL

In some examples, the imaging solution has a storage life of up to a month. Once a month has passed, a fresh imaging solution must be prepared. Preferably, the imaging solution is stored away from light. Preferably, the imaging solution is stored at a temperature of 10° C. or below, or, 5° C. or below, or, 4° C. or below.

In some examples, the collection solution and imaging solution are combined to form one solution.

### Method of Coating the Cuvette in the Composition

The present invention also comprises a method for coating a cuvette in the composition. The method includes the following steps:
(a) providing a cuvette comprising a collection chamber and an analysis chamber;
(b) providing a collection solution;
(c) providing an imaging solution;
(d) coating (completely or partially) the collection chamber of the cuvette with the collection solution;
(e) coating (completely or partially) the analysis chamber of the cuvette with the imaging solution;
(f) drying the coating of the collection solution on the collection chamber; and
(g) drying the coating of the imaging solution on the analysis chamber.

Before coating the cuvette with either the collection solution or the imaging solution, the storage life of each solution is checked.

In some examples, the collection solution is mixed thoroughly prior to coating the collection chamber of the cuvette with the collection solution. Preferably, the collection solution is mixed until it is homogeneous. The collection solution is then dispensed onto the collection chamber with a pipette. Preferably, from 10 to 20 μL, or, from 12.5 to 17.5 μL, or, 15 μL of the collection solution is dispensed onto the collection chamber. Preferably, the collection solution dispensed onto the collection chamber of the cuvette is taken from the middle of the collection solution.

In some examples, the imaging solution is mixed thoroughly prior to coating the analysis chamber of the cuvette with the imaging solution. Preferably, the imaging solution is mixed until it is homogeneous. The imaging solution is then dispensed onto the analysis chamber with a pipette. Preferably, from 10 to 20 μL, or, from 12.5 to 17.5 μL, or, 15 μL of the imaging solution is dispensed onto the analysis chamber. Preferably, the imaging solution dispensed onto the analysis chamber of the cuvette is taken from the middle of the imaging solution.

In some examples, a multichannel pipette is used to dispense the imaging solution onto the analysis chamber. Preferably, the multichannel pipette is adjusted so that from 1 to 60% by surface area, or, from 10 to 50% by surface area, or, from 20 to 40% by surface area, or, from 30 to 40% by surface area, or, 35% by surface area of the second chamber of the cuvette is coated with the imaging solution. Preferably, the multichannel pipette is adjusted so that from 40 to 99% by surface area, or, 50 to 90% by surface area, or, 80 to 40% by surface area, or from 70 to 60% by surface area, or 65% by surface area of the analysis chamber is not coated in the imaging solution. Preferably, the multichannel pipette is adjusted so that the imaging solution is placed at the first end of the analysis chamber and the second, closed, end of the analysis chamber with a portion of the analysis chamber present between the first end of the analysis chamber and the second, closed, end of the analysis chamber where no imaging solution is dispensed. Preferably, the proportion of imaging solution present at the first end of the analysis chamber is greater than the proportion of imaging solution present at the second, closed, end of the analysis chamber. Preferably, the ratio of the imaging solution present at the first end of the analysis chamber to the imaging solution present at the second, closed, end of the analysis chamber is from 5:3 to 5:4. Preferably, the analysis chamber has four opposing sidewalls, and the imaging solution is present on at most one of the four opposing sidewalls. Preferably, the analysis chamber has four opposing sidewalls, and the imaging solution is present on at most one of the four opposing sidewalls wherein the imaging solution is present on the sidewall at the first (open) end of the analysis chamber, present on the sidewall at the second (closed) end of the analysis chamber and not present at a middle region of the sidewall between the first (open) end and second (closed) end of the analysis chamber.

In an alternative example, a single pipette is used to dispense the imaging solution onto the analysis chamber. Preferably, the single pipette is adjusted so that from 1 to 60% by surface area, or, from 10 to 50% by surface area, or, from 20 to 40% by surface area, or, from 30 to 40% by surface area, or, 35% by surface area of the second chamber of the cuvette is coated with the imaging solution. Preferably, the single pipette is adjusted so that from 40 to 99% by surface area, or, 50 to 90% by surface area, or, 80 to 40% by surface area, or from 70 to 60% by surface area, or 65% by surface area of the analysis chamber is not coated in the imaging solution. Preferably, the single pipette is adjusted so that the imaging solution is placed at the first end of the analysis chamber and the second, closed, end of the analysis chamber with a portion of the analysis chamber present between the first end of the analysis chamber and the second, closed, end of the analysis chamber where no imaging solution is dispensed. Preferably, the proportion of imaging solution present at the first end of the analysis chamber is greater than the proportion of imaging solution present at the second, closed, end of the analysis chamber. Preferably, the ratio of the imaging solution present at the first end of the analysis chamber to the imaging solution present at the second, closed, end of the analysis chamber is from 5:3 to 5:4. Preferably, the analysis chamber has four opposing sidewalls, and the imaging solution is present on at most one of the four opposing sidewalls. Preferably, the analysis chamber has four opposing sidewalls, and the imaging solution is present on at most one of the four opposing sidewalls wherein the imaging solution is present on the sidewall at the first (open) end of the analysis chamber, present on the sidewall at the second (closed) end of the analysis chamber and not present at a middle region of the sidewall between the first (open) end and second (closed) end of the analysis chamber.

In some examples, the collection solution and collection chamber are dried after the collection solution is dispensed onto the collection chamber. Preferably, drying is conducted by exposing the collection solution and collection chamber to heat. Preferably, the collection solution and collection chamber are exposed to heat at a temperature of from 40 to 80° C., or, from 50 to 70° C., or, 60° C. Preferably, the collection solution and collection chamber are exposed to heat for a time period of from 1 to 20 minutes, or, from 5 to 15 minutes, or, 10 minutes.

In some examples, the imaging solution and analysis chamber are dried after the imaging solution is dispensed onto the analysis chamber. Preferably, drying is conducted by exposing the imaging solution and analysis chamber to heat. Preferably, the imaging solution and analysis chamber are exposed to heat at a temperature of from 40 to 80° C., or, from 50 to 70° C., or, 60° C. Preferably, the imaging solution and analysis chamber are exposed to heat for a period of from 1 to 20 minutes, or, from 5 to 15 minutes, or 10 minutes.

In some examples, the collection solution and imaging solution are dried onto the collection chamber and analysis chamber at the same time. Preferably, drying is conducted by exposing the collection solution, imaging solution, collection chamber and analysis chamber to heat. Preferably, the collection solution, imaging solution, collection chamber and analysis chamber are exposed to heat at a temperature of from 40 to 80° C., or, from 50 to 70° C., or, 60° C. Preferably, the collection solution, imaging solution, collection chamber and analysis chamber are exposed to heat for a period of from 1 to 20 minutes, or, from 5 to 15 minutes, or 10 minutes.

In some examples, the coated cuvette can be stored in a dust-free environment. Preferably, the storage temperature is from 10 to 30° C., or, from 15 to 25° C. Preferably, the relative humidity of storage is from 20 to 70% relative humidity, or, from 30 to 60% relative humidity.

In some examples, the cuvette is formed from two halves which are fixed together to form the cuvette. By providing the cuvette in two halves, the collection chamber and analysis chamber can be accessed easily during the process of coating the collection chamber and analysis chamber. Advantageously, this allows the formation of a homogeneous coating of the imaging solution in the analysis chamber.

### Method of Using the Composition-Coated Cuvette

The composition-coated cuvette can be used in the optical analysis of samples, such as a blood sample. To optically analyse a sample, the following steps are followed:
(a) place the sample inside the cuvette;
(b) mix a sample with a collection solution;
(c) introduce the mixture from step 1 to an imaging solution;
(d) subject the mixture from step 2 to a centrifugal force;
(e) expose the mixture to optical radiation; and
(f) obtain an optical image of the mixture.

In some examples, part of the sample is taken from the middle of the sample prior to step (b).

In some examples, the collection solution is present on the collection chamber of the cuvette during step (b), and step (b) is performed in the collection chamber of the cuvette. In some examples, the imaging solution is present on the analysis chamber of the cuvette during step (c), and step (c) is performed in the analysis chamber of the cuvette.

In some examples, the mixture is subjected to a centrifugal force in an Entia Liberty™ device during step (d). Preferably, the mixture is subjected to a centrifugal force of from 1000 to 2000 rpm, or, 1500 rpm. Preferably, the mixture is subjected to a centrifugal force at room temperature. Preferably, the mixture is subjected to a centrifugal force at from 15 to 40° C., or from 16 to 30° C., or from 18 to 25° C., or from 20 to 22° C., or at 20° C. Preferably, the mixture is subjected to a centrifugal force for from at most 30 minutes, or, at most 25 minutes, or, at most 20 minutes, or, at most 15 minutes, or, at most 10 minutes. Preferably, the mixture is subjected to a centrifugal force in an Entia Liberty™ device that provides a centrifugal force of 1500 rpm, at room temperature and for at most 20 minutes.

In some examples, the mixture is exposed to optical radiation from an LED light source at a wavelength of 460 nm during step (e). Preferably, the mixture is exposed to optical radiation at room temperature. Preferably, the mixture is exposed to optical radiation for at most 15 minutes, or, at most ten minutes, or, at most 5 minutes. Preferably, the mixture is exposed to optical radiation from an LED light source at a wavelength of 460 nm for at most 5 minutes and at room temperature.

In some examples, steps (e) and (f) are performed whilst the sample is within the cuvette.

In some examples, the optical image is analysed to translate the pixel count of the image obtained in step (f) into cell count.

### EXAMPLES

The following are non-limiting examples that discuss, with reference to tables and figures, at least some of the advantages of the present invention. The examples set forth herein are non-limiting examples and are merely examples among other possible examples.

### Example 1: Making the Composition

In the following non-limiting example, a composition comprising a collection solution and an imaging solution was made. The collection solution and imaging solution were made separately.

In this non-limiting example, a collection solution comprising polyvinylpyrrolidone, potassium oxalate, ethylenediaminetetraacetic acid dipotassium salt dehydrate, pluronic P-123 and water was made. The collection solution was made from stock solutions of each component.

In this non-limiting example, an imaging solution comprising acridine orange and water was made. The imaging solution was made from a stock solution of acridine orange.

The stock solutions for each component used in the collection and imaging solutions were made separately. To make the stock solutions, each component of the collection and imaging solutions were separately weighed in separate disposable trays and then mixed with the respective volume of water in separate clean and empty vials. Once each component of the collection and imaging solutions had been added to water, the concentration of each stock solution was verified by weighing the tray to ensure that no residue was left behind (the maximum amount of residue left behind was 0.005 g).

Any reagent that was a powder was completely dissolved in the water by stirring thoroughly.

The component pluronic P-123 was initially a gel block. To dissolve pluronic P-123 in water, the pluronic P-123 was stirred at 800 rpm for ten minutes at a room temperature of from 15 to 25° C. and relative humidity of 30 to 60% RH. The dilution increased with higher temperatures, though the temperature was carefully monitored to ensure that it did not go over 35° C.

Table 1 sets out the concentration of each stock solution made.

TABLE 1
The concentration (conc.) of each stock solution used to
make the collection solution and the imaging solution.

| Component present in each | Conc. in water |
|---|---|
| separate stock solution | (mg/mL) |
| Polyvinylpyrrolidone | 50.00 |
| Potassium oxalate | 50.00 |
| Ethylenediaminetetraacetic acid | 100.00 |
| dipotassium salt dehydrate | |
| Pluronic P-123 | 0.01 |
| Acridine orange | 1.00 |

The collection solution was made by combining the stock solutions for polyvinylpyrrolidone, potassium oxalate, ethylenediaminetetraacetic acid dipotassium salt dehydrate, pluronic P-123 and water to achieve the concentrations set out in Table 2. Once the stock solutions were combined, the solution was mixed so that the resultant collection solution was homogeneous throughout.

The imaging solution was made by diluting with water the stock solution of acridine orange to the desired concentration. Once the stock solution had been diluted, the solution was mixed so that the resultant imaging solution was homogeneous throughout.

The concentration of each component in the collection solution and imaging solution is tabulated in Table 2.

TABLE 2
The concentration (conc.) of each component used in
the collection solution and the imaging solution.
Each component was sourced from the stock solutions.

| | | |
|---|---|---|
| | | Conc. in each |
| Component in each solution | Function | solution (mg/mL) |

Collection solution

| | | |
|---|---|---|
| Polyvinylpyrrolidone | Agglutination agent | 10.00 |
| Potassium oxalate | Anticoagulant agent | 6.00 |
| Ethylenediaminetetraacetic acid | Anticoagulant agent | 7.00 |
| dipotassium salt dehydrate | | |
| Pluronic P-123 | Wetting agent | 0.10 |

Imaging solution

| | | |
|---|---|---|
| Acridine orange | Imaging agent | 0.02 |

Once formed, the collection solution and the imaging solution were kept separate (i.e. not mixed) until use.

### Example 2: Coating the Collection Chamber of the Cuvette with the Collection Solution

In the following non-limiting example, the collection chamber of the cuvette was coated with the collection solution. The cuvette was formed from two halves which were fixed together to form the cuvette.

In this non-limiting example, the following method was followed to coat the collection chamber of the cuvette half in the collection solution:
(a) the collection solution was stirred;
(b) 15 μL of the collection solution was drawn from the middle of the collection solution with a pipette;

(c) the 15 μL of the collection solution from step (b) was dispensed onto the surface of the collection chamber of the cuvette half within the accepted limits depicted in FIG. 1 and described below; and,
(d) the collection solution and collection chamber of the cuvette half were then dried.

FIG. 1 shows a collection chamber (160) of a cuvette half with a coating of the collection solution (150). FIG. 1 shows two images (110, 120) having acceptable coatings of the collection solution. As shown in FIG. 1, acceptable limits in which the collection solution is dispensed onto the collection chamber is for the entire collection chamber to be coated (110) or for the entire length of the collection chamber to be coated (120). By placing the collection solution within the acceptable limits, easy collection of the sample is ensured. FIG. 1 also shows two images (130, 140) having unacceptable coatings of the collection solution. As shown in FIG. 1, an unacceptable limit is for the collection solution to not cover the first end and the second end of the collection chamber (130). The collection solution needs to be present in areas where it can aid the collection of a sample and help maintain the integrity of the sample. By not covering the first end and/or second end of the collection chamber with the collection solution, the collection solution cannot perform this function. A second unacceptable limit is for the collection solution to extend over the second end of the collection chamber (140). By extending over the second end of the collection chamber with the collection solution, the collection solution blocks air vents present in the cuvette and thus prevents the sample from entering the analysis chamber: the sample will only enter the collection chamber.

To dry the collection solution and collection chamber, the collection solution and collection chamber were exposed to heat at a temperature of 60° C. for 10 minutes in a universal oven. During the drying step, the cuvette was not stacked on top of other cuvettes and was protected from dust particle contamination.

Once the collection solution was coated onto the collection chamber of the cuvette half, an imaging solution can be coated onto the analysis chamber of the cuvette half. Alternatively, an imaging solution can be coated onto the analysis chamber of the cuvette half prior to a collection solution being coated onto the collection chamber of the cuvette half. Once the collection and analysis chambers of the cuvette half had been coated with the collection and imaging solutions respectively, the two cuvette halves were fixed together to form a complete cuvette.

### Example 3: Coating the Analysis Chamber of the Cuvette with the Imaging Solution

In the following non-limiting example, the analysis chamber of the cuvette was coated with the imaging solution. The cuvette was formed from two halves which were fixed together to form the cuvette. One half had indentations on its inner surfaces that formed three of four of the opposing side walls of the analysis chamber; only the middle wall of these three sidewalls was partially coated with the imaging solution. The other two walls and other cuvette half were not coated. The other cuvette half was substantially smooth or flat. The indentations on the inner surface of the one cuvette half were so that, when the two halves were fixed together the collection chamber and analysis chamber existed within the main body of the cuvette. By providing the cuvette in two halves, the analysis chamber could be accessed easily during the process of coating.

In this non-limiting example, the following method was followed to coat the analysis chamber of the cuvette in the imaging solution:
(a) the imaging solution was stirred;
(b) a multichannel pipette, VIAFLO multichannel pipette, was turned on;
(c) the half of the cuvette to be coated was placed in the coating jig of the multichannel pipette (an area of the multichannel pipette designated for a surface to be coated to be placed);
(d) GRIPTIP tips were inserted into the multichannel pipette in the below order (with (4) being closest to the first end of the analysis chamber and (1) being closest to the second, closed end, of the analysis chamber), for a total of eight tips; FIG. 2 provides a depiction of the following ordering of the GRIPTIPS;
   (1) 2 tips (210);
   (2) 3 spaces (220);
   (3) 6 tips (230);
   (4) 5 spaces (240);
(e) the GRIPTIPS were submerged into the imaging solution and 3 μL of the imaging solution was collected by the GRIPTIPS;
(f) the GRIPTIPS were visually inspected to ensure that the collected volume of imaging solution was even for all the GRIPTIPS;
(g) the GRIPTIPS were aligned above the coating jig and lowered so as to gently touch the surface of the middle wall of the three sidewalls of the analysis chamber of the cuvette half;
(h) each GRIPTIP dispensed a first droplet containing 0.94 μL imaging solution onto the surface;
(i) the GRIPTIPS were realigned by moving the GRIPTIPS 2 mm in the direction of the second, closed end, of the analysis chamber;
(j) each GRIPTIP dispensed a second droplet containing 0.94 μL imaging solution onto the surface; and
(k) the imaging solution and analysis chamber of the cuvette half were then dried.

Once the pipettes had dispensed the second droplet (step (j)), the cuvette half and GRIPTIPS were removed from the coating jig. The GRIPTIPS were placed above absorbent tissue and any remaining sample was removed from the pipettes.

FIG. 3 provides an image of the cuvette half (300) with a collection chamber (340) and an analysis chamber (350). In FIG. 3, the areas of the analysis chamber (350) coated with the imaging solution are highlighted. As shown in FIG. 3, the parts of the analysis chamber (350) coated by the imaging solution are labelled 310 and 330, and the part of the analysis chamber not coated by the imaging solution is labelled 320. The length of the two parts of the analysis chamber coated by the imaging solution is 22 mm (310) and 10 mm (330). The total area of the analysis chamber coated with the imaging solution is 35% by surface area with 15 μL of the imaging solution.

To dry the imaging solution and the analysis chamber, the imaging solution and analysis chamber were exposed to heat at a temperature of 60° C. for 10 minutes in a universal oven. During the drying step, the cuvette was not stacked on top of other cuvettes and was protected from dust particle contamination. Advantageously, coating the imaging solution onto the analysis chamber ensures that the imaging solution comes into contact with the sample during the entire time of centrifugation. As a result, coating the imaging solution onto the analysis chamber reduces variation in absorption of the imaging solution by the sample. The imaging solution is present in the analysis chamber and does not enter the collection chamber owing to the imaging solution being dried onto the surface of the analysis chamber.

Once the imaging solution was coated onto the analysis chamber of the cuvette half, a collection solution can be coated onto the collection chamber of the cuvette half. Alternatively, a collection solution can be coated onto the collection chamber of the cuvette half prior to the imaging solution being coated onto the analysis chamber of the cuvette half. Once the collection and analysis chambers of the cuvette half had been coated with the collection and imaging solutions respectively, the two cuvette halves were fixed together to form a complete cuvette.

### Example 4: Coating the Analysis Chamber of the Cuvette with the Imaging Solution

In the following non-limiting example, the analysis chamber of the cuvette was coated with the imaging solution. The cuvette was formed from two halves which were fixed together to form the cuvette. One half had indentations on its inner surfaces that formed three of four of the opposing side walls of the analysis chamber; only the middle wall of these three sidewalls was partially coated with the imaging solution. The other two walls and other cuvette half were not coated. The other cuvette half was substantially smooth or flat. The indentations on the inner surface of the one cuvette half were so that, when the two halves were fixed together the collection chamber and analysis chamber existed within the main body of the cuvette. By providing the cuvette in two halves, the analysis chamber could be accessed easily during the process of coating.

In this non-limiting example, the following method was followed to coat the analysis chamber of the cuvette in the imaging solution:
(a) the imaging solution was stirred;
(b) a single pipette was provided;
(c) the single pipette was submerged into the imaging solution and 15 μL of the imaging solution was collected by the single pipette;
(d) the single pipette was placed over the surface of the middle wall of the three sidewalls of the analysis chamber of the cuvette half and the 15 μL of the imaging solution was dispensed in drops across the surface so as to achieve a coating of the analysis chamber wherein the total area of the analysis chamber coated with the imaging solution is 35% by surface area, and wherein the coating was on one of four of the sidewalls that form the analysis chamber, and the coating of the imaging solution was present only at the end of the sidewall at the first (open) end of the analysis chamber and at the end of the sidewall at the second (closed) end of the analysis chamber, and not present at a middle region between the first (open) end and second (closed) end of the analysis chamber;
(f) the imaging solution and analysis chamber of the cuvette half were then dried.

To dry the imaging solution and the analysis chamber, the imaging solution and analysis chamber were exposed to heat at a temperature of 60° C. for 10 minutes in a universal oven. During the drying step, the cuvette was not stacked on top of other cuvettes and was protected from dust particle contamination. Advantageously, by coating the imaging solution onto the analysis chamber ensures that the imaging solution comes into contact with the sample during the entire time of centrifugation: coating the imaging solution onto the analysis chamber reduces variation in absorption of the imaging solution by the sample. The imaging solution is present in the analysis chamber and does not enter the collection chamber owing to the imaging solution being dried onto the surface of the analysis chamber.

Once the imaging solution was coated onto the analysis chamber of the cuvette half, a collection solution can be coated onto the collection chamber of the cuvette half. Alternatively, a collection solution can be coated onto the collection chamber of the cuvette half prior to the imaging solution being coated onto the analysis chamber of the cuvette half. Once the collection and analysis chambers of the cuvette half had been coated with the collection and imaging solutions respectively, the two cuvette halves were fixed together to form a complete cuvette.

### Example 5: Analysing the Effect of Concentration of the Agglutination Agent in a Solution

In the following non-limiting example, the effect of the agglutination agent present in a solution upon the image obtained was analysed. The agglutination agent analysed in this example was polyvinylpyrrolidone.

In this non-limiting example, a solution comprising polyvinylpyrrolidone, acridine orange and water was made. The solution was made from a stock solutions of polyvinylpyrrolidone and acridine orange. The stock solutions were made as set out in Example 1.

Three different solutions having three different concentrations of polyvinylpyrrolidone were made. The three different concentrations of polyvinylpyrrolidone were: 10.00, 20.00 and 30.00 mg/mL. The solution having a concentration of the polyvinylpyrrolidone at 10.00 mg/mL was labelled Solution 1, the solution having a concentration of the polyvinylpyrrolidone at 20.00 mg/mL was labelled Solution 2, and, the collection solution having a concentration of the polyvinylpyrrolidone at 30.00 mg/mL was labelled Solution 3. The solutions were then made by combining the stock solutions for polyvinylpyrrolidone, acridine orange and water to achieve the concentrations set out in Table 3. Once the stock solutions were combined, the solutions were mixed so that the resultant solution was homogeneous throughout.

TABLE 3
The concentration of each component in the solutions.

| | Conc. of each component in the | | |
|---|---|---|---|
| Component in each | collection solutions (mg/mL) | | |
| collection solution | Solution 1 | Solution 2 | Solution 3 |
| Polyvinylpyrrolidone | 10.00 | 20.00 | 30.00 |
| Acridine orange | 0.02 | 0.02 | 0.02 |

Glass capillary tubes (Microslide 0.3×0.5 mm glass capillary tubes) were coated in the solutions. Different glass capillary tubes were used for the different solutions. The glass capillary tubes were coated by the following method:
(a) the solution was stirred;
(b) 50 μL of the solution was drawn from the middle of the solution with a pipette;
(c) the 50 μL of the solution from step (b) was dispensed onto the glass capillary tube; and,
(d) the solution and glass capillary tube were dried.

To dry the solution onto the glass capillary tube, the solution and glass capillary tube were exposed to heat at a temperature of 60° C. for 10 minutes in a universal oven.

Once the glass capillary tubes has been coated in the solutions, a sample of venous blood was then inserted into a glass capillary tube coated with Solution 1. The glass capillary tube along with the blood sample was placed into a laboratory centrifuge operating at 3000G for 5 minutes at room temperature. During the centrifugation, the blood sample separated into the platelet, agranulocyte, granulocyte and red blood cell layers. A Nikon D5100 camera took an image of the separated blood sample. The image was produced with an LED light operating at a wavelength of 460 nm at room temperature. The same procedure was performed for each glass capillary tube coated with Solution 2 or Solution 3. The experiment was then repeated for sixteen samples.

The results of the analysis are shown in FIG. 4. The image labelled 410 corresponds to Solution 1, the image labelled 420 corresponds to Solution 2 and the image labelled 430 corresponds to Solution 3.

### Example 6: Analysing the Effect of the Concentration of the Anticoagulant Agent in a Solution

In the following non-limiting example, the effect of the anticoagulant agent in a solution upon the image obtained was analysed. The anticoagulant agent analysed in this example was ethylenediaminetetraacetic acid dipotassium salt.

In this non-limiting example, a solution comprising ethylenediaminetetraacetic acid dipotassium salt dehydrate, polyvinylpyrrolidone, acridine orange and water was made. The solution was made from stock solutions of each component. The stock solutions were made as set out in Example 1. The concentration of the stock solution containing polyvinylpyrrolidone was made so that polyvinylpyrrolidone was present at a concentration of 3.0 mg/mL, and the concentration of the stock solution containing acridine orange was made so that acridine orange was present at a concentration of 0.02 mg/mL

Two different solutions having two different concentrations of ethylenediaminetetraacetic acid dipotassium salt dehydrate were analysed. The two different concentrations of ethylenediaminetetraacetic acid dipotassium salt dehydrate were: 7.00 and 19.00 mg/ml. The solution having a concentration of the ethylenediaminetetraacetic acid dipotassium salt dehydrate at 7.00 mg/mL was labelled Solution 4 and the solution having a concentration of the ethylenediaminetetraacetic acid dipotassium salt dehydrate at 19.00 mg/mL was labelled Solution 5. The solutions were then made by combining the stock solutions for ethylenediaminetetraacetic acid dipotassium salt dehydrate, polyvinylpyrrolidone, acridine orange and water to achieve the concentrations set out in Table 4. Once the stock solutions were combined, the solutions were mixed so that the resultant solution was homogeneous throughout.

TABLE 4
The concentration of each component in the solutions

| | |
|---|---|
| | Conc. of each component in the |
| Component in each | collection solutions (mg/mL) |

| collection solution | Solution 4 | Solution 5 |
|---|---|---|
| Ethylenediaminetetraacetic | 7.00 | 19.00 |
| acid dipotassium salt | | |
| dehydrate | | |
| Polyvinylpyrrolidone | 10.00 | 10.00 |
| Acridine orange | 0.20 | 0.20 |

Glass capillary tubes (Microslide 0.3×0.5 mm glass capillary tubes) were coated in the solutions. Different glass capillary tubes were used for the different solutions. The glass capillary tubes were coated by the following method:
(a) the solution was stirred;
(b) 50 μL of the solution was drawn from the middle of the solution with a pipette;
(c) the 50 μL of the solution from step (b) was dispensed onto the glass capillary tube; and,
(d) the solution and glass capillary tube were dried.

To dry the solution onto the glass capillary tube, the solution and glass capillary tube were exposed to heat at a temperature of 60° C. for 10 minutes in a universal oven.

Once the glass capillary tubes has been coated in the solutions, a sample of venous blood was then inserted into a glass capillary tube coated with Solution 4. The glass capillary tube along with the blood sample was placed into a laboratory centrifuge operating at 3000G for 5 minutes at room temperature. During the centrifugation, the blood sample separated into the platelet, agranulocyte, granulocyte and red blood cell layers. A Nikon D5100 camera took an image of the separated blood sample. The image was produced with an LED light operating at a wavelength of 460 nm at room temperature. The same procedure was performed for each glass capillary tube coated with Solution 5. The experiment was then repeated for twelve samples.

The results showed that solutions comprising a high concentration of ethylenediaminetetraacetic acid dipotassium salt dehydrate damaged cells in the sample analysed.

### Example 7: Comparing the Composition of the Present Invention with a Different Composition (not According to the Present Invention)

In this non-limiting example, a solution similar to the composition of the present invention coated on a cuvette is compared to a cuvette coated with a different composition (not according to the present invention).

The solution not according to the present invention was the solution described in U.S. Pat. No. 6,365,104B1. The solution described in U.S. Pat. No. 6,365,104B1 comprises sodium heparin, ethylenediaminetetraacetic acid dipotassium salt dehydrate, acridine orange and potassium oxalate. Table 5 sets out the mass of each component in the solution from U.S. Pat. No. 6,365,104B1, and the corresponding concentrations.

TABLE 5
The mass of each component present in the solution in U.S.
Pat. No. 6,365,104B1, and the corresponding concentrations.

| | Mass in | |
|---|---|---|
| | U.S. Pat. No. 6,365,104 | Concentration |
| Reagent | (mg) | (mg/mL) |
| Potassium oxalate | 0.3000 | 6.000 |
| Sodium heparin | 3.8 USP/ml | 76.0 USP/ml |
| Ethylenediaminetetraacetic | 0.4400 | 8.800 |
| acid | | |
| dipotassium salt dehydrate | | |
| Acridine orange | 0.0056 | 0.112 |

A solution replicating the solution in U.S. Pat. No. 6,365,104B1 was then made with the following concentrations: potassium oxalate at 6.000 mg/mL, sodium heparin at 76.0 USP/ml, ethylenediaminetetraacetic acid dipotassium salt dehydrate at 7.0 mg/mL and polyvinylpyrrolidone at 10 mg/mL, and, acridine orange at 0.110 mg/mL. Polyvinylpyrrolidone was included in the composition replicating the composition in U.S. Pat. No. 6,365,104B1 because polyvinylpyrrolidone was present in the composition of the present invention.

The solution similar to the composition of the present invention was then made with the following concentrations present in the solution: potassium oxalate at 6.000 mg/mL, ethylenediaminetetraacetic acid dipotassium salt dehydrate at 7.0 mg/mL, polyvinylpyrrolidone at 10 mg/mL, and, acridine orange at 0.110 mg/mL.

The solutions were made from stock solutions as set out in Example 1. The solutions were then made by combining the stock solutions for each component so as to achieve the above concentrations. Once the stock solutions were combined, the solutions were mixed so that the resultant collection solution was homogeneous throughout.

Glass capillary tubes (Microslide 0.3×0.5 mm glass capillary tubes) were then coated in the solutions. Different glass capillary tubes were used for the different solutions. The glass capillary tubes were coated by the following method:
(a) the solution was stirred;
(b) 50 μL of the solution was drawn from the middle of the solution with a pipette;
(c) the 50 μL of the solution from step (b) was dispensed onto the glass capillary tube; and,
(d) the solution and glass capillary tube were dried.

To dry the solution onto the glass capillary tube, the solution and glass capillary tube were exposed to heat at a temperature of 60° C. for 10 minutes in a universal oven.

Once the glass capillary tubes has been coated in the solutions, a sample of venous blood was then inserted into a glass capillary tube coated with the solution from U.S. Pat. No. 6,365,104B1. The glass capillary tube along with the blood sample was placed into a laboratory centrifuge operating at 3000G for 5 minutes at room temperature. During the centrifugation, the blood sample separated into the platelet, agranulocyte, granulocyte and red blood cell layers. A Nikon D5100 camera took an image of the separated blood sample. The image was produced with an LED light operating at a wavelength of 460 nm at room temperature. The same procedure was performed for a glass capillary tube coated with the solution similar to the composition of the present invention.

The results of the analysis are shown in FIG. 5. The solution from U.S. Pat. No. 6,365,104B1 is labelled 510 and the solution similar to the composition of the present invention is labelled 520. As shown in FIG. 5, the solution similar to the composition of the present invention provides improved clarity of the images obtained of the blood sample.

### Example 8: Analysing the Effect of Placing the Imaging Solution at the First End and Second, Closed, End (and not the Middle) of the Analysis Chamber

In this non-limiting example, the effect of placing the imaging solution towards or at the first (open) end and towards or at the second (closed) end (and not a middle portion) of the analysis chamber was analysed.

In this non-limiting example, the collection and imaging solutions were made as set out in Example 1, and a cuvette was coated as set out in Examples 2 and 3. The cuvette was formed from two halves which were fixed together to form the cuvette.

A further cuvette was coated with the collection and imaging solutions. The cuvette was formed from two halves which were fixed together to form the cuvette. One half had indentations on its inner surfaces that formed three of four of the opposing side walls of the collection and analysis chambers. The other half was substantially smooth or flat, and formed the fourth opposing side wall of the collection and analysis chambers. The collection chamber was coated with the collection solution as set out in Example 2. The analysis chamber of the cuvette was then coated with the imaging solution. The coating of the imaging solution on the cuvette half having indentations was not restricted to being present at only the first (open) end and second (closed) end, and not a middle portion, of the middle wall of the three sidewalls of the analysis chamber of the cuvette half (as described in Example 3). By not restricting the location of the imaging solution coating on the cuvette half, a portion of the imaging solution could be trapped between the cuvette halves when two halves of the cuvette were fixed together. Therefore, when a blood sample enters the analysis chamber, the portion of the imaging solution trapped cannot enter the blood sample and instead remains trapped between the two halves of the cuvette.

A venous blood sample was then inserted into each of the assembled cuvettes, and each cuvette along with the blood samples were placed in an Entia Liberty™ device separately. For each cuvette and blood sample, the device was run for 20 minutes at 1500 rpm at room temperature. The sample separated into the platelet, agranulocyte, granulocyte and red blood cell layers. Still in the Entia Liberty™ device, the blood samples were then exposed to an LED light source operating at a wavelength of 460 nm for five minutes at room temperature and an image was obtained of the sample.

FIG. 6 shows images (610, 620 and 630) obtained for cuvettes that had a coating of the imaging solution not restricted to being present at only the first (open) end and second (closed) end, and not a middle portion, of the middle wall of the three sidewalls of the analysis chamber of the cuvette half. As can be seen in FIG. 6, light beams are present in the image obtained.

Advantageously, by placing the imaging solution towards or at the first (open) end and towards or at the second (closed) end (and not a middle portion) of the middle wall of the three sidewalls of the analysis chamber of the cuvette half, the light beams are removed from the desired image of the buffy coat. The part of the analysis chamber not coated with the imaging solution corresponds to the position of the buffy coat layer of the separated blood sample. The process of coating the cuvette is a manual process and it cannot be ensured that there will be no coating on the sidewalls, and therefore the location of the imaging solution on the middle wall of the three sidewalls was restricted. The light beams (in 610, 620 and 630) correspond to imaging solution trapped by the adhesive during the fixing process used to fix together the two cuvette halves. By (a) placing the imaging solution only on the middle wall of the three sidewalls of the analysis chamber of the cuvette half having indentations, as well as (b) not placing the imaging solution in a middle portion of the middle wall of the analysis chamber, light beams in the image obtained of the sample can be removed or reduced.

Further advantageously, the imaging solution is coated on the analysis chamber. The imaging solution therefore has contact with the biological sample during the entire centrifuge and imaging process, thus allowing the mechanism by which the imaging agent operates to work efficiently.

Further advantageously, by not coating the collection chamber with the imaging solution no residue of the imaging solution will be left in the collection chamber, resulting in less variability seen between images obtained.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

Although certain example aspects of the invention have been described, the scope of the appended claims is not intended to be limited solely to these examples. The claims are to be construed literally, purposively, and/or to encompass equivalents.

1. A composition for analysing biological samples comprising:
a collection solution; and
an imaging solution;
wherein the collection solution comprises:
an anticoagulant agent;
an agglutination agent; and,
a wetting agent.

2. The composition of claim 1, wherein the collection solution comprises:
an anticoagulant agent at from 5 to 25 mg/ml;
an agglutination agent at from 5 to 25 mg/mL; and
a wetting agent at from 0.05 to 0.25 mg/mL.

3. The composition of claim 1, wherein the collection solution and/or the imaging solution further comprise(s) water (optionally, wherein the water is distilled water), or, alcohol solvents.

4. The composition of claim 1, wherein the collection solution comprises a first anticoagulant agent and a second anticoagulant agent, wherein the first anticoagulant agent and the second anticoagulant agent are different.

5. The composition of claim 1, wherein the anticoagulant agent(s) is/are one or more of ethylenediaminetetraacetic acid dipotassium salt dehydrate, potassium oxalate, ammonium-potassium oxalate, heparin, citrate, hirudin or any combination thereof.

6. The composition of claim 1, wherein the agglutination agent is one or more of polyvinylpyrrolidone, proteolytic enzymes such as bromelain, pepsin and/or trypsin, polyethylene glycol or any combination thereof.

7. The composition of claim 2, wherein the wetting agent is one or more of a well-known surfactant (that is ionic, non-ionic and/or cationic), pluronic P-123, silwet L600, fatty alcohol ethoxylates, alkyl phenol ethoxylates, fatty acid alkoxylates or any combination thereof.

8. The composition of claim 1, wherein the collection solution comprises:
(a) an anticoagulant agent at from 5 to 25 mg/mL, or, from 7.5 to 20 mg/mL, or, from 10 to 15 mg/mL, or, 13 mg/L1; and/or,
(b) an agglutination agent at from 5 to 25 mg/mL, or, from 7.5 to 20 mg/mL, or, from 9 to 11 mg/mL, or, 10 mg/mL; and/or,
(c) a wetting agent at from 0.050 to 0.250 mg/mL, or, from 0.075 to 0.120 mg/mL, or, from 0.900 to 0.125 mg/mL, or, 0.1 mg/mL.

9. The composition of claim 1, wherein the collection solution comprises a first anticoagulant agent at from 2 to 10 mg/mL, or, from 4 to 8 mg/mL, or, from 5 to 7 mg/mL, or, at 6 mg/mL, and, a second anticoagulant agent at from 3 to 11 mg/mL, or, from 5 to 9 mg/mL, or, from 6 to 8 mg/mL, or, 7 mg/ml; wherein the first anticoagulant agent and the second anticoagulant agent are different.

10. The composition of claim 1, wherein the collection solution comprises, or consists of, ethylenediaminetetraacetic acid dipotassium salt dehydrate, potassium oxalate, polyvinylpyrrolidone, pluronic P-123 and water; optionally wherein the water is distilled water.

11. The composition of claim 1, wherein the collection solution comprises:
(a) ethylenediaminetetraacetic acid dipotassium salt dehydrate agent at from 3 to 11 mg/mL, or, from 5 to 9 mg/mL, or, from 6 to 8 mg/mL, or, 7 mg/mL;
(b) potassium oxalate at from 2 to 10 mg/mL, or, from 4 to 8 mg/mL, or, from 5 to 7 mg/mL, or, 6 mg/mL;
(c) polyvinylpyrrolidone at from 5 to 25 mg/mL, or, from 7.5 to 20 mg/mL, or, from 9 to 11 mg/mL, or, 10 mg/mL; and,
(d) pluronic P-123 at from 0.050 to 0.250 mg/mL, or, from 0.075 to 0.120 mg/mL, or, from 0.900 to 0.125 mg/mL, or, 0.1 mg/mL;
the balance being water; optionally, wherein the water is distilled water.

12. The composition of claim 1, wherein the collection solution comprises ethylenediaminetetraacetic acid dipotassium salt dehydrate agent at 7 mg/mL; potassium oxalate at 6 mg/mL; polyvinylpyrrolidone at 10 mg/mL; and, pluronic P-123 at 0.1 mg/mL; the balance being water; optionally, wherein the water is distilled water.

13. The composition of claim 1, wherein the imaging solution comprises:
an imaging agent; optionally,
wherein the imaging solution further comprises water (optionally, wherein the water is distilled water), or, alcohol solvents.

14. The composition of claim 13, wherein the imaging agent is one or more of acridine orange, quaternary cationic metachromatic dyes such as Greifswalder's blue, blue borrel, rhodanile blue, toluylene blue, night blue, Hofmann's violet, basic orange 21, permanent dyes such as cell permanent cyanine dyes, SYTO dyes, oxayne dyes, phenanthridines (intercalating) dyes, indoles dyes, imidazole dyes or any combination thereof; and/or,
wherein the imaging agent has a concentration at from 0.01 to 0.030 mg/mL, or from 0.015 to 0.025 mg/mL, or from 0.019 to 0.021 mg/mL, or, 0.020 mg/mL.

15. The composition of claim 1, wherein the imaging solution comprises, or consists of, acridine orange and water; optionally, wherein the water is distilled water.

16. The composition of claim 1, wherein the imaging solution comprises acridine orange at from 0.01 to 0.030 mg/mL, or, from 0.015 to 0.025 mg/mL, or, from 0.019 to 0.021 mg/mL, or, 0.020 mg/mL; the balance being water; optionally, wherein the water is distilled water.

17. The composition of claim 1, wherein the imaging solution comprises acridine orange at 0.020 mg/mL; the balance being water; optionally, wherein the water is distilled water.

18. The composition of claim 1, wherein the imaging solution has a pH of from 3 to 6, or, from 3.5 to 4.5, or, 4.

19. The composition of claim 1, wherein the composition is a two-part composition, the collection solution and the imaging solution being separate prior to combining the collection solution and the imaging solution when analysing a biological sample; and/or,
wherein the collection solution and imaging solution are combined to form one solution.

20. A method of making the composition of claim 1, the method comprising the following steps:
(a) providing a collection solution; and
(b) providing an imaging solution;
wherein the collection solution comprises:
an anticoagulant agent;
an agglutination agent; and,
a wetting agent.

21. The method of making the composition of claim 20, wherein step (a) further includes the following steps:
(c) making a stock solution for the anticoagulant agent, the agglutination agent and the wetting agent; and
(d) combining the stock solutions.

22. The method of making the composition of claim 21, wherein step (b) further includes the following steps:
(e) providing an imaging agent;
(f) making a stock solution for the imaging agent; and
(g) diluting the stock solution of the imaging agent

23. The method of making the composition of claim 20, wherein the collection solution and imaging solution are as defined in claims 1 to 19.

24. The composition of claim 1, for use on a glass capillary tube or a cuvette; optionally, a disposable cuvette; and/or,
wherein the cuvette comprises a collection chamber and an analysis chamber, and,
wherein the collection chamber is coated (fully or partially) in the collection solution and the analysis chamber is coated (fully or partially) in the imaging solution.

25. The composition of claim 1, for use in the analysis of biological samples, optionally, a blood biological sample.
